(19) United States
(12) Patent Application Publication
(10) Pub. No.: US 2024/0423545 A1
(43) Pub. Date: Dec. 26, 2024

(54) NON-INVASIVE VITAL SIGNS MONITORING ON A MOBILE DEVICE USING AN UNDER-DISPLAY OPTICAL SENSOR

(71) Applicant: STMicroelectronics International N.V., Geneva (CH)

(72) Inventors: Alessandro GUMIERO, Milano (IT); Luigi DELLA TORRE, Lissone (IT); Charlotte MILANETTO, Grenoble (FR); Nicola PICOZZI, Nettuno (IT); David MAUCOTEL, Saint Cassien (FR); Benedicte VANGY, Grenoble (FR)

(21) Appl. No.: 18/339,480

(22) Filed: Jun. 22, 2023

(51) Int. Cl. A61B 5/00; A61B 5/024

(52) U.S. Cl. CPC A61B 5/6898; A61B 5/02416

(57) ABSTRACT

An example electronic device, computer-implemented method, and mobile device system for measuring the vital signs of a subject in a non-invasive manner are provided. The example electronic device may include a housing, an electronic display attached to the housing, and an optical sensor disposed within the housing. The electronic display may further include a first side configured to direct transmitted light toward a portion of a subject proximate the electronic display via organic light-emitting diodes (OLED). In addition, the optical sensor may be disposed, opposite the first side of the electronic display, and may be configured to receive reflected light off the portion of the subject. One or more vital signs of the subject may be determined based at least in part on the reflected light.

### TECHNOLOGICAL FIELD

Embodiments of the present disclosure relate generally to non-invasive vital signs monitoring devices, and more particularly, to non-invasive vital signs monitoring using photoplethysmography (PPG) techniques based on light received at an under-display sensor.

### BACKGROUND

Monitoring vital signs, such as heart rate, heart rate variability, blood pressure, and peripheral blood oxygen saturation (SpO2) in a non-invasive manner has become an important part of everyday routines. Many electronic devices such as watches, phones, fitness trackers, and tablets support monitoring of vital parameters in some capacity. Many non-invasive vital signs monitoring techniques utilize PPG techniques to determine vital signs, however, one of the most difficult problems for manufacturers of mobile devices is adding functionality, such as vital signs monitoring, without increasing the size and/or significantly increasing the power consumption of the electronic device.

Applicant has identified many technical challenges and difficulties associated with performing vital signs measurements on an electronic device using PPG techniques. Through applied effort, ingenuity, and innovation, Applicant has solved problems related to the use of PPG techniques to monitor vital signs on an electronic device by developing solutions embodied in the present disclosure, which are described in detail below.

### BRIEF SUMMARY

Various embodiments are directed to an example electronic device, computer-implemented method, and mobile device system for measuring the vital signs of a subject in a non-invasive manner. In some embodiments, the example electronic device may comprise a housing, an electronic display attached to the housing, and an optical sensor disposed within the housing. The electronic display may further comprise a first side configured to direct transmitted light toward a portion of a subject proximate the electronic display via organic light-emitting diodes (OLED). In addition, the optical sensor may be disposed, opposite the first side of the electronic display, configured to receive reflected light off the portion of the subject. In some embodiments, one or more vital signs of the subject may be determined based at least in part on the reflected light.

In some embodiments, no portion of the optical sensor may be visible from the first side of the electronic display.

In some embodiments, the reflected light may be light emitted from the electronic display via organic light-emitting diodes.

In some embodiments, the optical sensor may be enabled in an instance in which the electronic display is illuminated under the portion of the subject, and further the optical sensor may be disabled in an instance in which the electronic display is not illuminated under the portion of the subject.

In some embodiments, the optical sensor may receive a synchronization signal indicating a start of an electronic display refresh, and the optical sensor may be enabled based at least in part on the synchronization signal.

In some embodiments, the one or more vital signs may comprise at least a heart rate.

In some embodiments, the heart rate may be determined based at least in part on a period of the reflected light.

In some embodiments, the one or more vital signs may comprise at least one of heart rate variability and peripheral blood oxygen saturation.

In some embodiments, an ambient light vital signs measurement may be made by the optical sensor in an instance in which the electronic display is not illuminated, and the one or more vital signs of the subject may be determined based at least in part on the ambient light vital signs measurement.

In some embodiments, the optical sensor may be further configured to perform an ambient light operational measurement in an instance in which the optical sensor is not performing a vital signs measurement.

In some embodiments, an example computer-implemented method is further provided. The example computer-implemented method may comprise causing organic light-emitting diodes to transmit light from a first side of an electronic display toward a portion of a subject proximate the electronic display. The example computer-implemented method may further comprise receiving, from an optical sensor disposed within a housing opposite the first side of the electronic display, an electronic signal representative of reflected light off the portion of the subject. The example computer-implemented method may additionally comprise determining one or more vital signs of the subject based at least in part on the reflected light.

In some embodiments, no portion of the optical sensor is visible from the first side of the electronic display.

In some embodiments, the computer-implemented method may further comprise determining an instance in which the electronic display is on; and receiving, from the optical sensor, the electronic signal representative of the reflected light off the portion of the subject while the display is on.

In some embodiments, the computer-implemented method may further comprise receiving a synchronization signal indicting a start of an electronic display refresh, wherein determining the instance in which the electronic display is on is based at least in part on the synchronization signal.

In some embodiments, the computer-implemented method may further comprise receiving, from the optical sensor, the electronic signal representative of the reflected light off the portion of the subject, while the display is on proximate the portion of the subject.

In some embodiments, the computer-implemented method may further comprise receiving, from the optical sensor while the display is off, an ambient electronic signal representative of an ambient light in a surrounding environment and updating one or more vital signs of the subject based at least in part on the ambient light in the surrounding environment.

In some embodiments, the computer-implemented method may further comprise determining a period of the reflected light and calculating a heart rate based at least in part on the period of the reflected light.

In some embodiments, a mobile device system is further provided. The mobile device system may comprise a mobile device housing, an organic light-emitting diode (OLED) electronic display attached to the housing, an optical sensor disposed within the housing, and a host processor disposed within the housing and electrically connected to the optical sensor. In some embodiments, the OLED electronic display may comprise a first side configured to direct transmitted light toward a portion of a subject proximate the electronic display. In some embodiments, the optical sensor may be disposed opposite the first side of the electronic display and be configured to receive reflected light off the portion of the subject. In some embodiments, the host processor may further comprise a processor and an instruction memory including program code. The instruction memory and program code may be configured to, with the processor, cause the host processor to cause the electronic display to transmit light from the first side of the electronic display toward a portion of the subject proximate the electronic display; receive, from the optical sensor disposed within the housing opposite the first side of the electronic display, an electronic signal representative of the reflected light off the portion of the subject; and determine one or more vital signs of the subject based at least in part on the reflected light.

In some embodiments, no portion of the optical sensor may be visible from the first side of the electronic display.

In some embodiments, the host processor may be further configured to receive, from the optical sensor, the electronic signal representative of the reflected light off the portion of the subject in an instance in which the electronic display is illuminated under the portion of the subject, and determine one or more vital signs of the subject based at least in part on the electronic signal.

### BRIEF DESCRIPTION OF THE DRAWINGS

Reference will now be made to the accompanying drawings. The components illustrated in the figures may or may not be present in certain embodiments described herein. Some embodiments may include fewer (or more) components than those shown in the figures in accordance with an example embodiment of the present disclosure.

FIG. 1 depicts an example block diagram of an example vital signs measurement device in accordance with one or more embodiments of the present disclosure.

FIG. 2 depicts a block diagram of an example processing device in accordance with one or more embodiments of the present disclosure.

FIG. 3 illustrates an example mobile device comprising a vital signs measurement device in accordance with one or more embodiments of the present disclosure.

FIG. 4 illustrates a cross-section view of an example vital signs monitoring device monitoring the vital signs of a subject in accordance with one or more embodiments of the present disclosure.

FIG. 5 illustrates an example vital signs monitoring device receiving ambient light in accordance with one or more embodiments of the present disclosure.

FIG. 6 illustrates an example graph of reflected light in an example vital signs monitoring device in accordance with one or more embodiments of the present disclosure.

FIG. 7 depicts an example vital signs monitoring device utilizing an example synchronization signal to enable an optical sensor in accordance with one or more embodiments of the present disclosure.

FIG. 8 depicts an example flow chart of a process for determining one or more vital signs of a subject in accordance with one or more embodiments of the present disclosure.

FIG. 9 depicts an example flow chart of a process for updating one or more vital signs measurements based on ambient light in accordance with one or more embodiments of the present disclosure.

### DETAILED DESCRIPTION

Example embodiments will be described more fully hereinafter with reference to the accompanying drawings, in which some, but not all embodiments of the inventions of the disclosure are shown. Indeed, embodiments of the disclosure may be embodied in many different forms and should not be construed as limited to the embodiments set forth herein; rather, these embodiments are provided so that this disclosure will satisfy applicable legal requirements. Like numbers refer to like elements throughout.

Various example embodiments address technical problems associated with performing non-invasive vital signs measurements on a mobile electronic device. As understood by those of skill in the field to which the present disclosure pertains, there are numerous example scenarios in which a person may desire to perform non-invasive vital signs measurements using a mobile device.

For example, monitoring vital parameters, such as heart rate, heart rate variability, blood pressure, and peripheral blood oxygen saturation (SpO₂) in a non-invasive manner has become an important part of everyday health routines. Many electronic devices such as watches, phones, fitness trackers, and tablets support monitoring of vital parameters in some capacity.

Many non-invasive vital signs monitoring techniques utilize photoplethysmography (PPG) techniques to determine vital signs. PPG generally involves illuminating the skin or tissue with a light source and then detecting changes in the reflected light with a photodiode. As the volume of blood in the blood vessels changes due to blood being pumped through the blood vessels, so does the amount of light absorbed and/or reflected by the tissue. Many vital signs measurements may be made leveraging these properties. For example, changes in the light received at a photodiode may be used to indicate the heart rate and heart rate variability of the individual based on the photodiode readings. Similarly, the oxygen saturation of the blood effects the amount and color of light absorbed by the tissue. Photodiode readings may further indicate the peripheral blood oxygen saturation (SpO₂) of an individual based on the reflected light.

One of the most difficult problems for manufacturers of mobile devices is to add functionality, such as vital signs monitoring, without significantly changing the physical parameters of the mobile device. For example, manufacturers seek to avoid increasing the physical size of the device when adding additional functionality. In addition, manufacturers seek to avoid significant increases in power consumption when adding additional device functionality.

One approach to adding non-invasive vital signs monitors to a mobile device is to use dedicated hardware. Dedicated hardware may involve adding a chip, sensor, light, or other hardware device to the mobile device. Adding dedicated hardware to the mobile device is likely to change the form factor, potentially resulting in a bulkier device. Dedicate hardware is also likely to consume more power, hastening the drainage of power in a mobile device battery.

Another approach utilizes existing devices and sensors (e.g., the selfie camera on a smart phone) to monitor vital signs. While utilizing existing hardware on the mobile device does not generally require changes to the form factor and may only minimally increase the power consumption of the mobile device, solutions for measuring vital signs using existing hardware are often inaccurate and unreliable. Thus, there is a need for mobile device manufacturers to add functionality, such as non-invasive vital signs monitoring, without adding to the size of the mobile device, requiring external hardware, or substantially increasing the power consumption of the device.

The various example embodiments described herein utilize various techniques to obtain vital signs measurements in a non-invasive manner on a mobile electronic device. The vital signs measurement device described herein utilizes an optical sensor positioned under the screen of the electronic device to obtain vital signs measurements using PPG techniques. As described herein, the optical sensor positioned under the screen may capture light reflected off a subject's tissue. The reflected light may then be analyzed by a processing device to determine the vital signs of a subject using PPG techniques. In some embodiments, the non-invasive vital signs measurement device may utilize the light from the electronic display of the mobile device to illuminate the subject's tissue and subsequently measure various vital signs, including heart rate, heart rate variability, blood pressure, and SpO₂. In some embodiments, the placement of the optical sensor under the screen of the mobile device, hides the optical sensor from visibility from the front of the mobile device, thus, reflected light passes through the electronic display before it is captured by the optical sensor. Placing the optical sensor under the screen enables vital sign measurement capabilities to be added to a mobile electronic device without changing the form factor of the mobile electronic device. In addition, by utilizing the light from the electronic display, (such as an OLED screen) any increase in power consumption by the mobile device in order to perform vital signs measurements is minimal.

As further described herein, in some embodiments, the capture of reflected light may be synchronized with the refresh of the electronic display of the mobile device. For example, the vital signs monitoring mechanism may receive one or more screen refresh signals associated with the refresh of the mobile device electronic display. In such an embodiment, the sensor may only detect changes in reflected light received at the optical sensor in an instance in which the mobile device electronic display is illuminated directly above the optical sensor. In this way, noise from ambient light or the light of the mobile device screen surrounding the subject's tissue is reduced.

Further, in some embodiments, the optical sensor used to monitor changes in the light reflected off a subject's tissue may additionally be used to monitor ambient light when not utilized for vital signs monitoring. Measurements of ambient light may be utilized to adjust certain parameters of the mobile device, for example, the brightness of the screen. Using the optical sensor as an ambient light sensor may remove the need for additional sensors, thus reducing the space occupied within the mobile device and perhaps even the power consumed in supporting additional sensors.

As a result of the herein described example embodiments and in some examples, the accuracy of non-invasive monitors on mobile devices may be greatly improved. Further, the addition of the non-invasive vital signs measurement device according to the embodiments described herein may have a minimal impact on the form factor and power consumption of the mobile electronic device.

Referring now to FIG. 1, an example vital signs measurement device 100 is provided. As depicted in FIG. 1, the example vital signs measurement device 100 includes a processing device electrically connected to an electronic display 104. As further depicted in FIG. 1, the processing device 102 is electrically connected to an optical sensor 106.

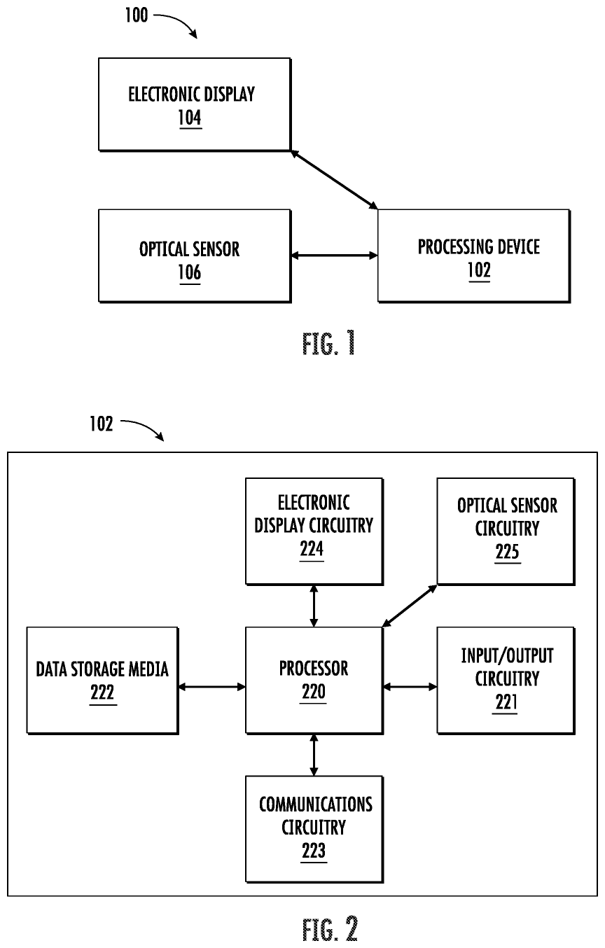

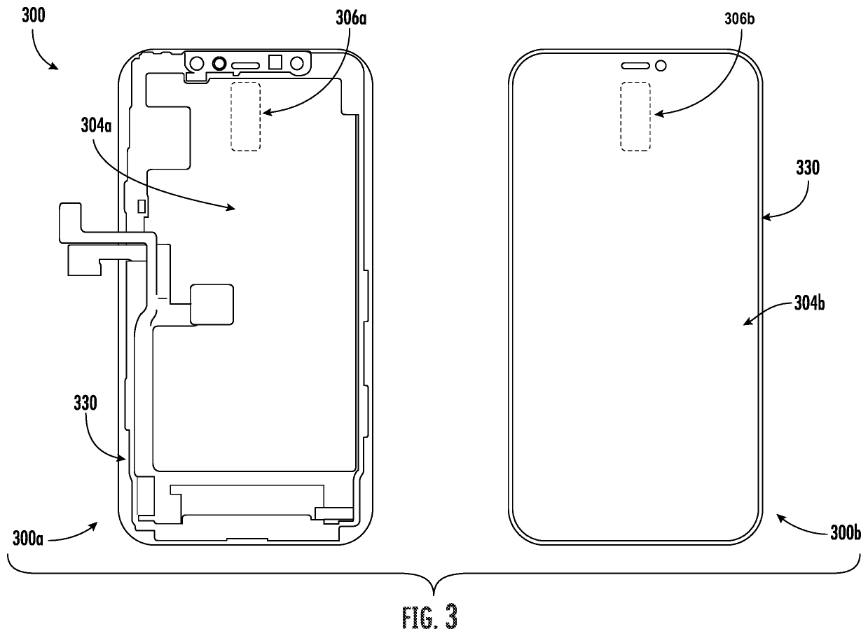

As depicted in FIG. 1, the vital signs measurement device 100 includes a processing device 102. A processing device 102 may include any processor, machine, microcontroller, or other electronic device configured to receive electronic data related to the light received by an optical sensor 106 and determine vital signs of a person or subject based on the received electronic data. As further depicted in FIG. 1, the processing device 102 may be electrically connected to an electronic display. Such an electrical connection may enable the processing device 102 to control the output of the display. Similarly, such an electrical connection may enable a processing device 102 to receive synchronizing signals related to the refresh of the electronic display. Various aspects of an example processing device 102 are further described in relation to FIG. 2.

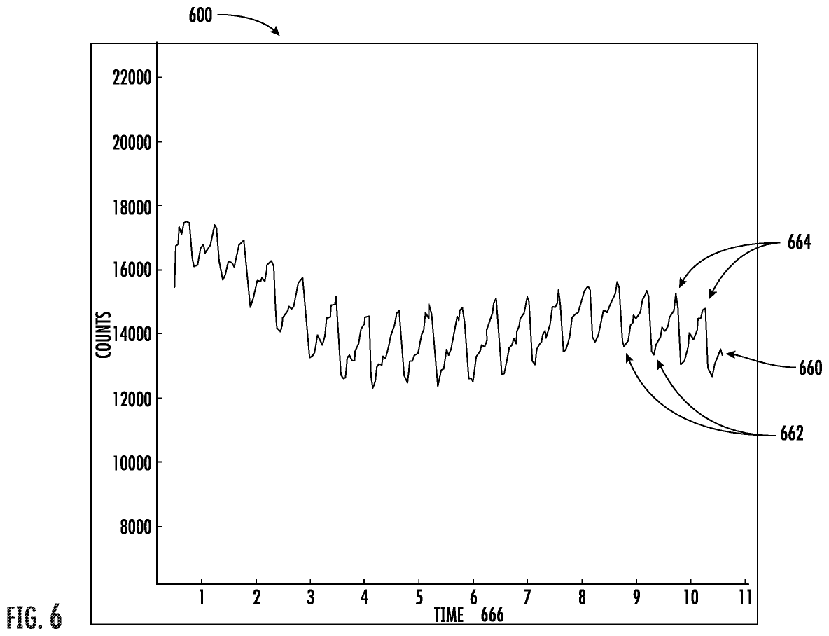

As further depicted in FIG. 1, the example vital signs measurement device 100 includes an optical sensor 106. An optical sensor 106 may be any device, sensor, photodiode, semiconductor device or other structure that produces an electric current corresponding to the intensity of light received at the optical sensor 106. In some embodiments, the optical sensor 106 may be configured to only sense light when the electronic display 104 is off. In addition, in some embodiments, the optical sensor 106 may be a super sensitive photodiode, boosted at blue wavelengths. These additional factors may enable the optical sensor 106 to be placed under the electronic display 104, invisible from the display side of the electronic display 104. In some embodiments, the optical sensor 106 may be a light sensitive semiconductor diode that creates an electron-hole pair at the p-n junction when a photon of sufficient energy strikes the diode. In this way, the electric current output by the optical sensor 106 may be proportional to the intensity of the light received at the optical sensor 106. For example, the electric current output by the optical sensor 106 may increase as the number of photons that strike the optical sensor 106 per second increases. In such an embodiment, the electric current output from the optical sensor 106 may be used to determine the intensity of light received at the optical sensor 106. As it relates to the determination of vital signs, in an instance in which a subject's finger is placed over the optical sensor 106, and light emitted from the electronic display 104 is reflected off the subject's finger, the intensity of light received at the optical sensor 106 may be proportional to the amount of light absorbed by the subject's finger, and thus, proportional to the amount or type of blood in the subject's finger. In such an instance, the electrical output from the optical sensor 106 may be used to determine certain vital signs, such as heart rate, heart rate variability, blood pressure, and peripheral blood oxygen saturation (SpO2). The graph 600 depicted in FIG. 6, further describes utilizing the output of the optical sensor 106 to determine vital signs. In some embodiments, the optical sensor 106 may comprise a color red-green-blue (RGB) ambient light sensor and proximity sensor, such as a VL6285.

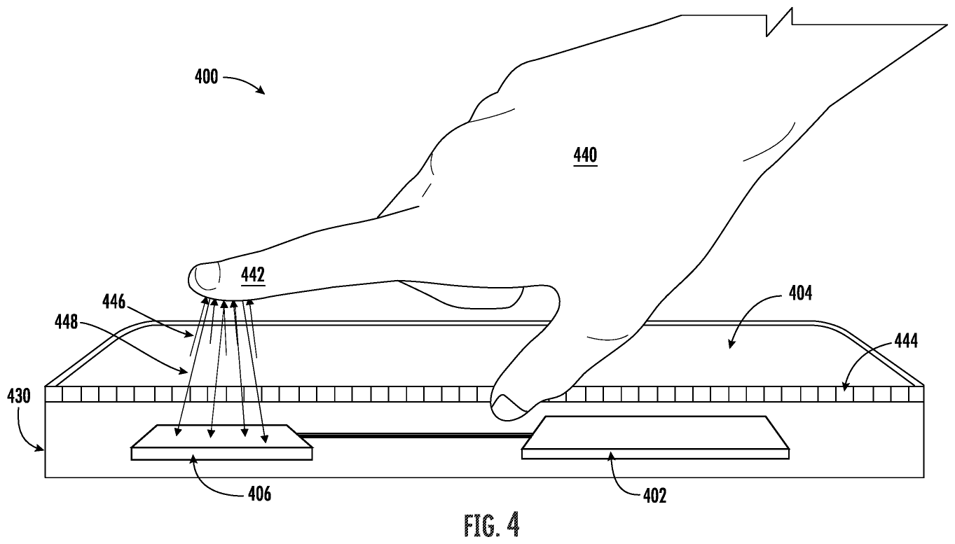

As further depicted in FIG. 1, the vital signs measurement device 100 includes an electronic display 104. An electronic display 104 may be any digital display, screen, monitor, or other device configured to output information in visual form based on a received electronic signal. An electronic display 104 may be transparent or semi-transparent to certain wavelengths of light, such that reflected light may be received by an optical sensor 106 behind or under the electronic display. Although primarily depicted as an organic light-emitting diode (OLED), the electronic display 104 may comprise an active-matrix OLED (AMOLED) display or other similar variation. In some embodiments, the electronic display 104 may comprise a plurality of pixels. Pixels may be the smallest unit of display in an electronic display. In some embodiments, each pixel of an electronic display may emit a red, green, and blue color at different intensities to output a specific color from the pixel. In some embodiments, for example in an OLED electronic display 104, each pixel may comprise an organic compound that emits light in response to an electric current. OLED pixels are described further in relation to FIG. 4.

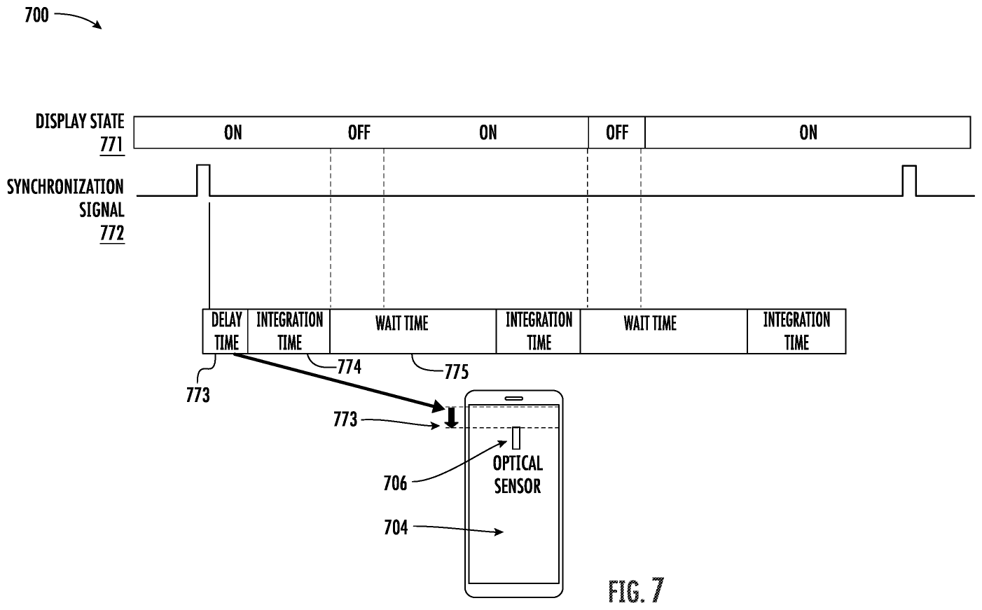

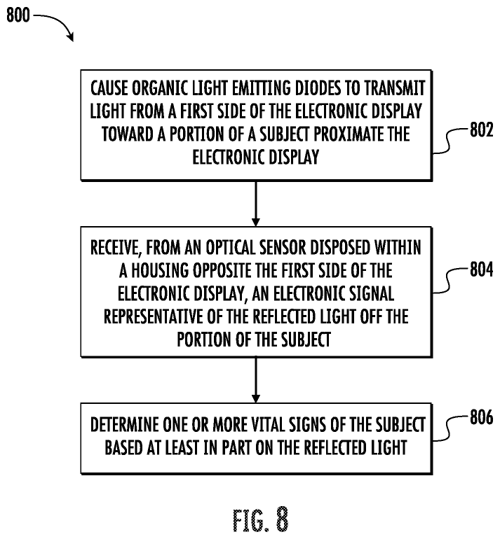

In some embodiments, the electronic display 104 may be configured to refresh the individual pixels at an established rate, for example, 60 hertz. In some embodiments, an electronic display 104 may refresh each pixel on the electronic display 104 during a refresh cycle. In some embodiments, the electronic display 104 may illuminate the pixels on the electronic display 104 one row at a time. In some embodiments, the refresh of the electronic display 104 may be signaled by a synchronization signal transmitted to various controllers and devices in electronic communication with the electronic display. Synchronization signals may enable coordination between modules based on the start of a refresh cycle of a display. For example, an optical sensor under an electronic display 104 may be configured to capture light data only when the electronic display 104 is disabled, in other words, the electronic display 104 is not illuminated. Synchronization signals are discussed in further detail in relation to FIG. 7.

Referring now to FIG. 2, FIG. 2 illustrates an example processing device 102 in accordance with at least some example embodiments of the present disclosure. The processing device 102 includes processor 220, input/output circuitry 221, data storage media 222, communications circuitry 223, electronic display circuitry 224, and optical sensor circuitry 225. In some embodiments, the processing device 102 is configured, using one or more of the sets of circuitry 220, 221, 222, 223, 224, and/or 225, to execute and perform the operations described herein.

Although components are described with respect to functional limitations, it should be understood that the particular implementations necessarily include the use of particular computing hardware. It should also be understood that in some embodiments certain of the components described herein include similar or common hardware. For example, two sets of circuitry may both leverage use of the same processor(s), network interface(s), storage medium(s), and/or the like, to perform their associated functions, such that duplicate hardware is not required for each set of circuitry. The user of the term “circuitry” as used herein with respect to components of the apparatuses described herein should therefore be understood to include particular hardware configured to perform the functions associated with the particular circuitry as described herein.

Particularly, the term “circuitry” should be understood broadly to include hardware and, in some embodiments, software for configuring the hardware. For example, in some embodiments, “circuitry” includes processing circuitry, storage media, network interfaces, input/output devices, and/or the like. Alternatively, or additionally, in some embodiments, other elements of the processing device 102 provide or supplement the functionality of other particular sets of circuitry. For example, the processor 220 in some embodiments provides processing functionality to any of the sets of circuitry, the data storage media 222 provides storage functionality to any of the sets of circuitry, the communications circuitry 223 provides network interface functionality to any of the sets of circuitry, and/or the like.

In some embodiments, the processor 220 (and/or co-processor or any other processing circuitry assisting or otherwise associated with the processor) is/are in communication with the data storage media 222 via a bus for passing information among components of the processing device 102. In some embodiments, for example, the data storage media 222 is non-transitory and may include, for example, one or more volatile and/or non-volatile memories. In other words, for example, the data storage media 222 in some embodiments includes or embodies an electronic storage device (e.g., a computer readable storage medium). In some embodiments, the data storage media 222 is configured to store information, data, content, applications, instructions, or the like, for enabling the processing device 102 to carry out various functions in accordance with example embodiments of the present disclosure.

The processor 220 may be embodied in a number of different ways. For example, in some example embodiments, the processor 220 includes one or more processing devices configured to perform independently. Additionally, or alternatively, in some embodiments, the processor 220 includes one or more processor(s) configured in tandem via a bus to enable independent execution of instructions, pipelining, and/or multithreading. The use of the terms “processor” and “processing circuitry” should be understood to include a single core processor, a multi-core processor, multiple processors internal to the processing device 102, and/or one or more remote or “cloud” processor(s) external to the processing device 102.

In an example embodiment, the processor 220 is configured to execute instructions stored in the data storage media 222 or otherwise accessible to the processor. Alternatively, or additionally, the processor 220 in some embodiments is configured to execute hard-coded functionality. As such, whether configured by hardware or software methods, or by a combination thereof, the processor 220 represents an entity (e.g., physically embodied in circuitry) capable of performing operations according to an embodiment of the present disclosure while configured accordingly. Alternatively, or additionally, as another example in some example embodiments, when the processor 220 is embodied as an executor of software instructions, the instructions specifically configure the processor 220 to perform the algorithms embodied in the specific operations described herein when such instructions are executed.

As one particular example embodiment, the processor 220 is configured to perform various operations associated with determining vital signs of a subject in a non-invasive way. For example, in some embodiments, the processor 220 includes hardware, software, firmware, and/or a combination thereof, that causes organic light-emitting diodes to transmit light from a first side of the electronic display (e.g., electronic display 104) toward a portion of a subject proximate the electronic display. Additionally, or alternatively, in some embodiments, the processor 220 includes hardware, software, firmware, and/or a combination thereof, that receives, from an optical sensor (e.g., optical sensor 106) disposed within a housing opposite the first side of the electronic display, an electronic signal representative of the reflected light off the portion of the subject. Additionally, or alternatively, in some embodiments, the processor 220 includes hardware, software, firmware, and/or a combination thereof, that determines one or more vital signs of the subject based at least in part on the reflected light. Additionally, or alternatively, in some embodiments, the processor 220 includes hardware, software, firmware, and/or a combination thereof, that determines an instance in which the electronic display is on; and receives, from the optical sensor, the electronic signal representative of the reflected light off the portion of the subject while the display is on.

In some embodiments, the processing device 102 includes input/output circuitry 221 that provides output to the user and, in some embodiments, to receive an indication of a user input. In some embodiments, the input/output circuitry 221 is in communication with the processor 220 to provide such functionality. The input/output circuitry 221 may comprise one or more user interface(s) (e.g., user interface) and in some embodiments includes a display that comprises the interface(s) rendered as a web user interface, an application user interface, a user device, a backend system, or the like. The processor 220 and/or input/output circuitry 221 comprising the processor may be configured to control one or more functions of one or more user interface elements through computer program instructions (e.g., software and/or firmware) stored on a memory accessible to the processor (e.g., data storage media 222, and/or the like). In some embodiments, the input/output circuitry 221 includes or utilizes a user-facing application to provide input/output functionality to a client device and/or other display associated with a user.

In some embodiments, the processing device 102 includes communications circuitry 223. The communications circuitry 223 includes any means such as a device or circuitry embodied in either hardware or a combination of hardware and software that is configured to receive and/or transmit data from/to a network and/or any other device, circuitry, or module in communication with the processing device 102. In this regard, the communications circuitry 223 includes, for example in some embodiments, a network interface for enabling communications with a wired or wireless communications network. Additionally, or alternatively in some embodiments, the communications circuitry 223 includes one or more network interface card(s), antenna(s), bus(es), switch(es), router(s), modem(s), and supporting hardware, firmware, and/or software, or any other device suitable for enabling communications via one or more communications network(s). Additionally, or alternatively, the communications circuitry 223 includes circuitry for interacting with the antenna(s) and/or other hardware or software to cause transmission of signals via the antenna(s) or to handle receipt of signals received via the antenna(s). In some embodiments, the communications circuitry 223 enables transmission to and/or receipt of data from a client device in communication with the processing device 102.

The electronic display circuitry 224 includes hardware, software, firmware, and/or a combination thereof, that supports various functionality associated with configuring and/or communicating with an electronic display (e.g., electronic display 104). For example, in some embodiments, the electronic display circuitry 224 includes hardware, software, firmware, and/or a combination thereof to communicate with the electronic display according to an established protocol to provide appropriate configuration and/or calibration parameters and/or to receive synchronization signals. In some embodiments, synchronization may be utilized by an electronic device to indicate the start of a frame refresh. Such synchronization signals may enable a vital signs measurement device (e.g., vital signs measurement device 100) to determine an instance in which the electronic display 104 is on (enabled) or off (disabled). Additionally, or alternatively, in some embodiments, the electronic display circuitry 224 includes hardware, software, firmware, and/or a combination thereof, to control the output display on the electronic display. For example, the electronic display circuitry 224 may be configured to output a certain image or sequence of images during vital signs measurement. In some embodiments, the electronic display circuitry 224 may cause a certain color to be output to the electronic display, or a certain region of the electronic display to be illuminated. In some embodiments, the electronic display circuitry 224 includes a separate processor, specially configured field programmable gate array (FPGA), or a specially programmed application specific integrated circuit (ASIC).

The optical sensor circuitry 225 includes hardware, software, firmware, and/or a combination thereof, that supports various functionality associated with configuring and/or communicating with an optical sensor (e.g., optical sensor 106). For example, in some embodiments, the optical sensor circuitry 225 includes hardware, software, firmware, and/or a combination thereof to communicate with the optical sensor according to an established protocol to provide appropriate configuration and/or calibration parameters to receive accurate data representing received light at the optical sensor 106. Additionally, or alternatively, in some embodiments, the optical sensor circuitry 225 includes hardware, software, firmware, and/or a combination thereof, to coordinate with the electronic display, such that measurements from the optical sensor are recorded in coordination with the enabling and disabling of the electronic display. Thus, the optical sensor circuitry 225 may record reflected light for determination of a subject's vital signs when the electronic display is illuminated under the subject's finger and record ambient light when the electronic display is not illuminated. In some embodiments, the optical sensor circuitry 225 includes a separate processor, specially configured field programmable gate array (FPGA), or a specially programmed application specific integrated circuit (ASIC).

Additionally, or alternatively, in some embodiments, one or more of the sets of circuitry 220-225 are combinable. Additionally, or alternatively, in some embodiments, one or more of the sets of circuitry perform some or all of the functionality described associated with another component. For example, in some embodiments, one or more sets of circuitry 220-225 are combined into a single module embodied in hardware, software, firmware, and/or a combination thereof. Similarly, in some embodiments, one or more of the sets of circuitry, for example electronic display circuitry 224, and/or optical sensor circuitry 225, is/are combined such that the processor 220 performs one or more of the operations described above with respect to each of these circuitry individually.

Referring now to FIG. 3, FIG. 3 depicts a back side view 300a and a front side view 300b of an example mobile device system 300 comprising a vital signs measurement device. As shown in the back side view 300a, the mobile device system 300 comprises a housing 330 attached to an electronic display 304a and enclosing an optical sensor 306a. The optical sensor 306a as depicted is disposed on the back side of the electronic display 304a and is not visible from the front side view 300b. Although shown in FIG. 3 with a portion of the housing 330 removed, such that the back side of the electronic display 304a and the optical sensor 306a are visible, in some embodiments, the housing 330 completely encloses the optical sensor 306a, such that the optical sensor 306a is only visible from within the housing 330. As shown in the front side view 300b, the optical sensor 306b is behind the electronic display 304b and is not visible from the front side view 300b.

As depicted in FIG. 3, a vital signs measurement device (e.g., vital signs measurement device 100) may be included in a mobile device system 300. A mobile device system 300 may be any mobile electronic device, including a mobile phone, smart phone, watch, tablet, wearable, monitor, smart screen, or other similar device, configured to include an electronic display (e.g., electronic display 104, 304a, 304b). A mobile device system 300 may include additional components, such as a host processor, instruction memory, storage memory, input/output mechanisms, additional sensors, antennas, a battery, and other components necessary to the operation of the mobile device system 300.

As further depicted in FIG. 3, the mobile device system 300 may include a housing 330. The housing 330 may be any protective cover or casing configured to protect the internal components of the mobile device system 300 from damage due to handling, impact, the elements, or other destructive forces. In some embodiments, the housing 330 may provide a watertight seal to protect the internal components of the mobile device system 300 from water or other liquids. In some embodiments, the housing 330 may attach to the electronic display 304a/304b, for example, with an adhesive, or similar attaching mechanism. A housing 330 for a mobile device system 300 may comprise a plastic, wood, metal, polycarbonate, thermoplastic polyurethane, silicone, rubber, or any combination thereof, configured to protect the internal components of the mobile device system from damage, while allowing access to certain input/output components of the mobile device system 300. In some embodiments, the housing 330 together with the electronic display 304a/304b may fully enclose the internal components of the mobile device system 300, including the optical sensor 306a/306b, such that the internal components are not visible from without the housing 330. For example, the housing 330 may comprise the bottom/back of the mobile device system 300 and further enclose the sides, with one side (top/front) at least partially open. The electronic display 304a/304b may be positioned to fit within the opening of the housing 330 and attached to fully enclose the internal components, such as the optical sensor 306a/306b.

Referring now to FIG. 4, and example vital signs measurement device 400 is provided. As depicted in FIG. 4, the example vital signs measurement device 400 includes an electronic display 404 comprising a plurality of pixels 444, wherein the electronic display 404 is attached to a housing 430. As further depicted in FIG. 4, the housing 430 together with the electronic display 404 enclose an optical sensor 406 electrically connected to a processing device 402, both of which are disposed within the housing 430 and under the electronic display 404. FIG. 4 further depicts transmitted light 446 transmitted by the electronic display 404 and encountering a subject's finger 442. A portion of the transmitted light 446 is reflected toward the vital signs measurement device 400 as reflected light 448 and received by the optical sensor 406 on the side of the electronic display 404 opposite the subject 440.

As depicted in FIG. 4, the example vital signs measurement device includes an electronic display 404 comprising a plurality of pixels 444 configured to emit transmitted light 446. A pixel 444 may be any unit of display in an electronic display 404. A pixel 444 may be configured to output an intensity of light or a combination of light intensities based on an electronic signal indicating a desired output. For example, in some embodiments, each pixel of an electronic display 404 may emit a red, green, and blue color at different intensities to generate a specific color from the pixel 444. In some embodiments, for example in an OLED electronic display 404, each pixel 444 may comprise an organic compound that emits light in response to an electric current. An OLED pixel 444 may comprise a layer of organic material situated between an anode and a cathode. In an instance in which a voltage difference is applied to the anode and cathode, the movement of electrons and holes within the organic material due to the change in voltage results in the emission of light (e.g., transmitted light 446). The anode and cathode of the OLED pixel 444 may be selected based on the conductivity and transparency of the material.

As depicted in FIG. 4, an electronic display 404 may comprise a plurality of pixels 444 (e.g., OLED pixels). The plurality of pixels 444 may be illuminated in a coordinated manner to generate a display image. For example, in some embodiments, the pixels 444 may be illuminated one row at a time and move sequentially from one side of the display to the other. Due to the speed of illumination, the electronic display 404 may appear to be fully illuminated. In some embodiments, a synchronization signal may be sent to indicate the first row of the electronic display 404 may be illuminated.

As further depicted in FIG. 4, the electronic display 404 generates transmitted light 446. Transmitted light 446 may be any light comprising photons emitted from the electronic display 404 towards a subject 440, such as a user of the vital signs measurement device 400. In some embodiments, the transmitted light 446 may comprise a specific wavelength of light, for example, green light may be transmitted, or red light, depending on the types of vital signs measured. In some embodiments, the emitted light may comprise a spectrum of wavelengths, for example, white light, comprising light with a variety of wavelengths, may be transmitted. In some embodiments, the transmitted light 446 may comprise non-visible light, such that the light is not visible to humans. For example, in an instance in which the electronic display 404 is not in use, an infrared light source may be used to generate transmitted light 446. The transmitted light 446 may be directed out one side of the electronic display 404, for example, the side opposite the optical sensor 406.

In some embodiments, a subject 440 or user of the vital signs measurement device 400 may be proximate the side of the electronic display 404 configured to emit transmitted light 446. During operation of the vital signs measurement device 400 a portion of the transmitted light 446 may encounter a portion of the subject 440, such as the subject's finger 442 as depicted in FIG. 4. In an instance in which the transmitted light 446 encounters the subject's finger 442, a portion of the transmitted light 446 is absorbed by the subject's finger 442, while another portion of the transmitted light 446 is reflected back toward the electronic display 404 and the optical sensor 406. By utilizing the electronic display 404 to generate transmitted light 446, additional hardware, such as light-emitting diodes (LEDs) may be avoided. By avoiding additional LEDs, the form factor of the mobile device may remain unchanged, and changes in the power consumption of the device may be minimal. In addition, by using the electronic display 404 to generate transmitted light 446, there is no need for an additional blinking light which may be an annoyance to a subject 440 using the mobile device.

The amount of light reflected as reflected light 448 is dependent at least in part on the amount of blood in the tissue of the subject's finger 442 and the wavelength of the transmitted light 446. As a subject's 440 heart pumps, the arteries are distended as the amount of blood volume increases. In general, the increase in blood volume results in an increase in transmitted light 446 absorbed, and a decrease in reflected light 448 received at the optical sensor 406. As described further in relation to FIG. 6, the amount of reflected light 448 received at the optical sensor 406 may be used to determine vital signs, such as heart rate, heart rate variability, blood pressure, and SpO₂.

As further depicted in FIG. 4, the optical sensor 406 is placed under the electronic display 404, or in other words, on the side of the electronic display 404 opposite the subject 440. By placing the optical sensor 406 under the electronic display 404, the reflected light 448 resulting from the light from the electronic display 404 reflecting off a subject 440 may be used to determine vital signs of the subject. In addition, the form factor of the mobile device may not need to be changed based on the addition of a hardware sensor.

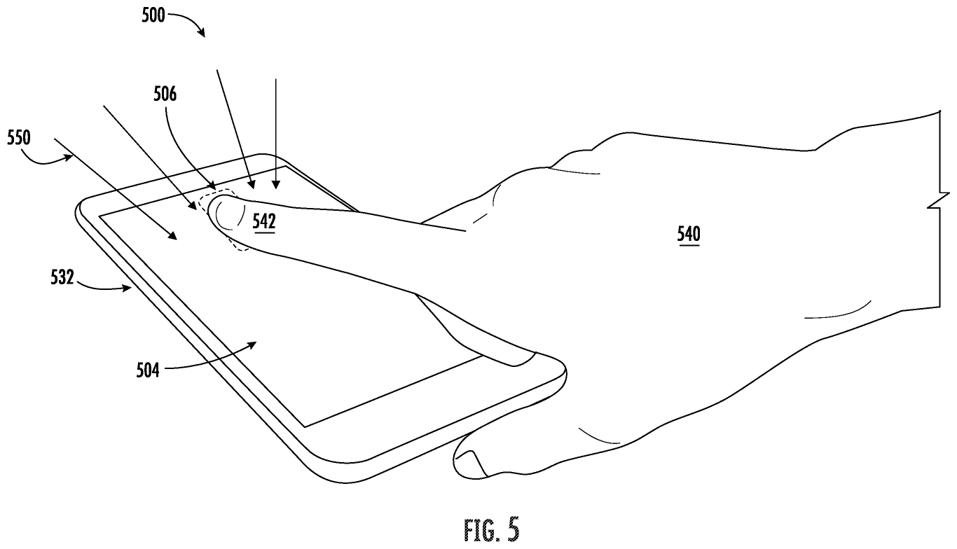

Referring now to FIG. 5, an example vital signs measurement device 500 is depicted. As depicted in FIG. 5, the example vital signs measurement device 500 includes an electronic display 504 attached to a housing 532. As further depicted in FIG. 5, the example vital signs measurement device 500 is configured to receive ambient light 550 at an optical sensor 506 under the electronic display 504, in an instance in which the electronic display 504 is off or disabled.

As depicted in FIG. 5, the example vital signs measurement device 500 may be configured to receive ambient light 550. Ambient light 550 may be any light received at the optical sensor 506 that is not emitted from the electronic display 504, for example, light from external sources. In some embodiments, ambient light 550 may be measured during the measurement of vital signs. For example, the vital signs measurement device 500 may determine an instance in which the electronic display 504 is off and capture the reading from the optical sensor 506. In such an instance, the ambient light 550 measurement may be utilized to adjust the vital signs measurement. For example, the vital signs measurement device 500 may determine variations in the received reflected light is due to a change in ambient light 550. By measuring the ambient light 550, an adjustment to the calculated vital signs may be made.

In some embodiments, the optical sensor 506 may measure the ambient light 550 in an instance in which no vital signs measurements are made. The level of ambient light 550 may be utilized to adjust certain parameters of the mobile device, for example, the brightness of the screen. By utilizing the optical sensor 506 to measure the ambient light 550, the optical sensor 506 may replace another sensor on the mobile device. Thus, utilizing the optical sensor 506 to perform multiple operations on the mobile device may enable physical space to be conserved.

Referring now to FIG. 6, an example graph 600 depicting the intensity of reflected light 660 (e.g., reflected light 448) received at an optical sensor (e.g., optical sensor 106, 306a, 306b, 406, 506) over a period of time 666 is provided. As depicted in FIG. 6, the intensity of the reflected light 660 varies periodically over time 666. The intensity of the reflected light 660 experiences regular local minimums 662 and local maximums 664. The intensity of the reflected light 660 may be utilized to determine certain vital signs. For example, as shown by graph 600, the heart rate may be determined by calculating the period of the intensity of the reflected light 660. As depicted, the local minimums 662 are generated when a larger volume of blood is in the subject's finger 542 such that the larger volume of blood absorbs more of the transmitted light (e.g., transmitted light 446) from the electronic display and less light is returned as reflected light (e.g., reflected light 448). Thus, the local minimums 662 may correspond with a heartbeat and the time between consecutive local minimums 662 may be used to determine a heart rate. For example, give the time at two consecutive local minimums 662 (t_{LM1}, t_{LM2}), a heart rate in beats per minute (BPM) may be calculated by the following equation:

$$rate ( BPM ) = \frac{1}{{t}_{LM 2} - {t}_{LM 1}} \cdot 60 ,$$

where the rate is the heart rate in beats per minute, t_{LM1} is the time of the first local minimum 662, and t_{LM2} is the time of the second local minimum 662. Thus, in an instance in which t_{LM1} is 8.8 seconds and t_{LM2} is 9.5 seconds, the calculated heart rate would be:

$$rate ( BPM ) = \frac{1}{{t}_{LM 2} - {t}_{LM 1}} \cdot 60 = \frac{1}{9.5 - 8.8} \cdot 60 = 85.7 BPM .$$

Similarly, an average heart rate over a period of time may be determined by counting the local minimums 662 over a fixed period of time. In addition, a heart rate variability may be determined by tracking the time between local minimums 662 over a period of time. The heart rate variability may quantify the change in time between heart beats over a specified period of time.

In some embodiments, a peripheral blood oxygen saturation (SpO₂) may be calculated. In general, the SpO₂ of a subject may be determined by calculating the ratio between oxygenated hemoglobin and de-oxygenated hemoglobin. In practice, oxygenated hemoglobin absorbs light differently than deoxygenated hemoglobin. Thus, the levels of oxygenated hemoglobin and deoxygenated hemoglobin may be determined based on the reflected light. In some embodiments, the reflected light from two different wavelengths of transmitted light may be used to determine the oxygenated hemoglobin and deoxygenated hemoglobin levels. The two wavelengths of transmitted light may comprise any combination of red light, green light, blue light, or infrared light, or any other two wavelengths of light configured to reflect based on the quantity of oxygenated hemoglobin. Utilizing the reflected light, a peripheral blood oxygen saturation measurement may be determined.

Referring now to FIG. 7, an example synchronization sequence 700 between the electronic display 704 and the optical sensor 706 is provided. As depicted in FIG. 7, an electronic display 704 and the associated display driver, may utilize various signals to coordinate the refresh of the electronic display 704. A synchronization signal 772 may be any electronic signal or series of electronic signals indicating the start of a refresh period of an electronic display 704. A refresh period of an electronic display 704 may be any period in which the content or output of the electronic display 704 is updated. In some embodiments, the pixels of the electronic display 704 may be illuminated one or more times between synchronization signals 772. For example, as shown in FIG. 7, the pixels of the electronic display 704 may be illuminated and re-illuminated a plurality of times between synchronization signal 772 assertions.

In some embodiments, during the determination of vital signs by a vital signs measurement device (e.g., vital signs measurement device 100, 400, 500), the measurements captured by the optical sensor 706 for purposes of determining vital signs, may be recorded in an instance in which the pixels directly above the optical sensor 706 are illuminated (e.g., display state 771 is “ON”). In such an instance, the transmitted light from the electronic display 704 illuminates the subject's finger and the reflected light is at a maximum. Recording measurements captured by the optical sensor 706 while the electronic display 704 is illuminated above the optical sensor 706 (and under the subject's finger), reduces the noise from other light sources and increases the accuracy of the vital signs measurements.

Timing of the recording of optical sensor 706 measurements, such that the optical sensor 706 measurements are determined in an instance in which the electronic display 704 is illuminated directly above the optical sensor 706, may be coordinated based on the synchronization signal 772. For example, as shown in FIG. 7, a delay time 773 may be determined from the time of receipt of the synchronization signal 772 based on the location of the optical sensor 706. The delay time 773 may be any time representing the time that elapses between the reception of the synchronization signal 772 and the illumination of the pixels of the electronic display 704 directly above the optical sensor 706. As shown in FIG. 7, during operation, the delay time 773 may represent the time the rows of the electronic display 704 at or near the top of the electronic display 704 are illuminated previous to the illumination of the pixels of the electronic display 704 directly above the optical sensor 706. In some embodiments, the delay time 773 may be determined during manufacturing of the electronic device based on the physical placement of the optical sensor 706 in the electronic device. For example, in an instance in which the optical sensor 706 is at or near the bottom of the electronic display 704, and the electronic display 704 refreshes starting at the top, the delay time 773 may be longer than in an instance in which the optical sensor 706 is placed at or near the top of the electronic display 704.

As further depicted in FIG. 7, a vital signs measurement device may also account for the integration time 774 of the optical sensor 706 to synchronize the recording of optical sensor 706 measurements with the electronic display 704. The integration time 774 may correspond with the length of time a particular optical sensor 706 measures the electric signal output corresponding with the light received at the optical sensor 706. To obtain accurate vital signs measurements, the entirety of the integration time 774 may occur during the time in which the display state 771 is “ON.” Thus, the delay time 773 may be adjusted such that the integration time 774 of the optical sensor 706 occurs while the pixels directly above the optical sensor 706 are illuminated.

As further depicted in FIG. 7, the wait time 775 represents the time between successive integration times 774 during a single refresh period. In some embodiments, the pixels of the electronic display 704 may illuminate in a rolling pattern. For example, in some embodiments, one row, or a group of rows, of an electronic display 704 may illuminate starting at the top of the display, then the next row or group of rows may illuminate, until all the rows have illuminated. The wait time 775 may be any time period representing the amount of time between successive illuminations of a row or group of rows of pixels. During operation, the wait time 775 may represent the amount of time required to illuminate all other rows of the electronic display 704 before returning to row or group of rows of pixels directly above the optical sensor 706.

In some embodiments, the optical sensor 706 may be utilized to measure ambient light (e.g., ambient light 550). As described in relation to FIG. 5, ambient light may be monitored during the determination of vital signs by a vital signs measurement device (e.g., vital signs measurement device 100, 400, 500). In addition, ambient light may be measured during other operations of the mobile device to determine parameters such as screen brightness, etc. In some embodiments, ambient light measurements may be performed in an instance in which the electronic display 704 is off directly above the optical sensor 706. Measuring ambient light in an instance in which the electronic display 704 is off, enables accurate measurements of the ambient light, minimally affected by the illumination of the electronic display 704. The synchronization signal 772 may be further utilized in order to ensure measurements of the ambient light are performed when the electronic display 704 is off directly above the optical sensor 706. To perform measurements at the optical sensor 706 while the electronic display is off (e.g., display state 771 is “OFF”), the delay time 773 may be adjusted such that the integration time 774 aligns with the display state 771 “OFF.” In some embodiments, the wait time 775 may also be adjusted to ensure the integration time 774 aligns throughout the refresh cycle.

Referring now to FIG. 8, an example process 800 for measuring the vital signs of a subject (e.g., subject 440, 540) in a non-invasive manner is provided. At block 802, a processing device (e.g., processing device 102, 402) may cause organic light-emitting diodes (e.g., pixels 444) to transmit light (e.g., transmitted light 446) from a first side (e.g., electronic display 304b) of the electronic display (e.g., electronic display 104, 404, 504, 704) toward a portion of a subject (e.g., subject's finger 442, 542) proximate the electronic display. As described herein, the electronic display may be configured to output transmitted light having a single wavelength and/or comprising multiple wavelengths. In some embodiments, an area, or portion of the electronic display, for example, a portion of the screen proximate the subject's finger, may be configured to illuminate. In some embodiments, the electronic display may indicate to the subject the location of the optical sensor, such that the subject may place their finger on or near the optical sensor.

At block 804, the processing device may receive, from an optical sensor (e.g., optical sensor 106, 406, 506, 706) disposed within a housing (e.g., housing 330, 432, 532) opposite the first side of the electronic display (e.g., electronic display 304a), an electronic signal representative of the reflected light (e.g., reflected light 448) off the portion of the subject. As described herein, the optical sensor may be positioned on the opposite side of the electronic display from which the transmitted light is emitted. In addition, the optical sensor may be hidden from the view of the subject. In an instance in which the transmitted light encounters a portion of the subject, a portion of the transmitted light may be absorbed while another portion of the transmitted light is reflected by the portion of the subject back toward the electronic display. The reflected light may pass through the electronic display where it is received by the optical sensor on the opposite side of the electronic display.

At block 806, the processing device may determine one or more vital signs of the subject based at least in part on the reflected light. As described in relation to FIG. 6, certain vital signs may be determined based on the intensity of the reflected light received at the optical sensor. For example, a heart rate of the subject may be determined based on the periodicity of the intensity of the light reflected by the subject and received at the optical sensor. In some embodiments, a heart rate variability may also be determined based on the periodicity of the reflected light. Further, in some embodiments, more than one wavelength of light may be transmitted and received to determine the peripheral blood oxygen saturation of the subject.

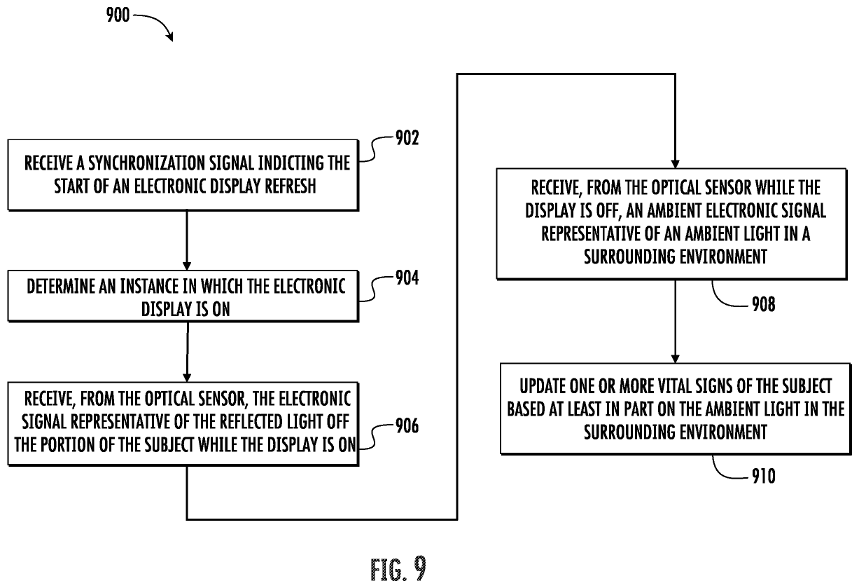

Referring now to FIG. 9, an example process 900 for synchronizing the measuring of the vital signs of a subject (e.g., subject 440, 540) with an electronic display (e.g., electronic display 104, 404, 504, 704) is further provided. At block 902, a processing device (e.g., processing device 102, 402) may receive a synchronization signal (e.g., synchronization signal 772) indicting the start of an electronic display refresh. As described herein, an electronic display refresh may update the displayed output of an electronic display. In addition, an electronic display may illuminate the pixels on the electronic display one or more times during an electronic display refresh. In some embodiments, the electronic display may illuminate and re-illuminate in a rolling pattern. Meaning, the row, or a group of rows, at the top of the electronic display may illuminate first. The next row, or group of rows may then be illuminated, until the last or bottom row or group of rows is illuminated. The electronic display may then return to the top and re-illuminate the row or group of rows at the top of the electronic display. The synchronization signal, in some embodiments, may indicate the start of the electronic display refresh and the illumination of the row or group of rows at the top of the electronic display.

At block 904, the processing device may determine an instance in which the electronic display is on. As described in relation to FIG. 7, the synchronization signal may be utilized to determine the precise time at which the pixels immediately above (or proximate) the optical sensor are illuminated. Determining the precise time may include determining the delay time (e.g., delay time 773) from the receipt of the synchronization signal until the time the pixels proximate the optical sensor are illuminated. The synchronization process may further include aligning the integration time (e.g., integration time 774) of the optical sensor with the on time of the proximate pixels. Further, a wait time (e.g., wait time 775) may be determined based on the time required to refresh all pixels on the electronic display and return to the pixels proximate the optical display. By determining the delay time, the integration time, and the wait time, the collection of measurements at the optical sensor may be synchronized with the illumination of pixels proximate the optical sensor throughout the refresh cycle of the electronic display.

At block 906, the processing device may receive, from the optical sensor (e.g., optical sensor 106, 406, 506, 706), the electronic signal representative of the reflected light (e.g., reflected light 448) off the portion of the subject (e.g., subject's finger 442, 542) while the display is on. By synchronizing the measurement of the optical sensor with the illumination of pixels proximate the optical sensor, the reflected light from the portion of the subject may be at a maximum, increasing the accuracy of the vital signs measurements.

At block 908, the processing device may receive from the optical sensor while the display is off, an ambient electronic signal representative of an ambient light (e.g., ambient light 550) in a surrounding environment. As further described in relation to FIG. 7, a synchronization signal may be utilized to measure the ambient light in a surrounding environment. A vital signs measurement device may be synchronized with the electronic display to measure the electrical output of the optical sensor at an instance in which the pixels proximate the optical sensor are not illuminated. Thus, a measure of the ambient light from external sources in the surrounding environment, may be determined.

At block 910, the processing device may update one or more vital signs of the subject based at least in part on the ambient light in the surrounding environment. In some embodiments, the ambient light measurements, and particularly, changes in the ambient light may be utilized to adjust the vital signs measurement. For example, the intensity of the ambient light received may be subtracted from the reflected light received at the optical sensor at a time in which the display is illuminated directly under the portion of the subject. In some embodiments, utilizing the ambient light measurements, may enable the processing device to attribute a change in the intensity of reflected light received to an increase, or decrease, in ambient light from the surrounding environment. Thus, in such circumstances, the vital signs measurements may be adjusted to compensate for the change in ambient light.

While this detailed description has set forth some embodiments of the present invention, the appended claims cover other embodiments of the present invention which differ from the described embodiments according to various modifications and improvements. For example, one skilled in the art may recognize that such principles may be applied to any electronic device utilizing reflected light to make determinations. For example, various health monitors, such as watches, phones, bands, etc. and also, various proximity sensors using reflected light to determine proximity or distance of an object.

Within the appended claims, unless the specific term “means for” or “step for” is used within a given claim, it is not intended that the claim be interpreted under 35 U.S.C. 112, paragraph 6.

Use of broader terms such as “comprises,” “includes,” and “having” should be understood to provide support for narrower terms such as “consisting of,” “consisting essentially of,” and “comprised substantially of” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

1. An electronic device comprising:
a housing;
an electronic display attached to the housing comprising:
a first side configured to direct transmitted light toward a portion of a subject proximate the electronic display via organic light-emitting diodes (OLED); and
an optical sensor disposed within the housing, opposite the first side of the electronic display, and configured to receive reflected light off the portion of the subject;
wherein one or more vital signs of the subject are determined based at least in part on the reflected light.

2. The electronic device of claim 1, wherein no portion of the optical sensor is visible from the first side of the electronic display.

3. The electronic device of claim 1, wherein the reflected light is light emitted from the electronic display via organic light-emitting diodes.

4. The electronic device of claim 3, wherein the optical sensor is enabled in an instance in which the electronic display is illuminated under the portion of the subject, and
wherein the optical sensor is disabled in an instance in which the electronic display is not illuminated under the portion of the subject.

5. The electronic device of claim 4, wherein the optical sensor receives a synchronization signal indicating a start of an electronic display refresh, and
wherein the optical sensor is enabled based at least in part on the synchronization signal.

6. The electronic device of claim 1, wherein the one or more vital signs comprise at least a heart rate.

7. The electronic device of claim 6, wherein the heart rate is determined based at least in part on a period of the reflected light.

8. The electronic device of claim 1, wherein the one or more vital signs comprises at least one of heart rate variability and peripheral blood oxygen saturation.

9. The electronic device of claim 1, wherein an ambient light vital signs measurement is made by the optical sensor in an instance in which the electronic display is not illuminated, and the one or more vital signs of the subject are determined based at least in part on the ambient light vital signs measurement.

10. The electronic device of claim 1, wherein the optical sensor is further configured to perform an ambient light operational measurement in an instance in which the optical sensor is not performing a vital signs measurement.

11. A computer-implemented method comprising:
causing organic light-emitting diodes to transmit light from a first side of an electronic display toward a portion of a subject proximate the electronic display;
receiving, from an optical sensor disposed within a housing opposite the first side of the electronic display, an electronic signal representative of reflected light off the portion of the subject; and
determining one or more vital signs of the subject based at least in part on the reflected light.

12. The computer-implemented method of claim 11, wherein no portion of the optical sensor is visible from the first side of the electronic display.

13. The computer-implemented method of claim 11, further comprising:
determining an instance in which the electronic display is on; and
receiving, from the optical sensor, the electronic signal representative of the reflected light off the portion of the subject while the display is on.

14. The computer-implemented method of claim 13, further comprising:
receiving a synchronization signal indicting a start of an electronic display refresh,
wherein determining the instance in which the electronic display is on is based at least in part on the synchronization signal.

15. The computer-implemented method of claim 14, further comprising:
receiving, from the optical sensor, the electronic signal representative of the reflected light off the portion of the subject, while the display is on proximate the portion of the subject.

16. The computer-implemented method of claim 15, further comprising:
receiving, from the optical sensor while the display is off, an ambient electronic signal representative of an ambient light in a surrounding environment; and
updating one or more vital signs of the subject based at least in part on the ambient light in the surrounding environment.

17. The computer-implemented method of claim 11, further comprising:
determining a period of the reflected light; and
calculating a heart rate based at least in part on the period of the reflected light.

18. A mobile device system comprising:
a mobile device housing;
an organic light-emitting diode (OLED) electronic display attached to the housing comprising:
a first side configured to direct transmitted light toward a portion of a subject proximate the electronic display;
an optical sensor disposed within the housing, opposite the first side of the electronic display, and configured to receive reflected light off the portion of the subject; and
a host processor, disposed within the housing and electrically connected to the optical sensor, the host processor comprising:
a processor; and
an instruction memory including program code, the instruction memory and program code configured to, with the processor, cause the host processor to:
cause the electronic display to transmit light from the first side of the electronic display toward a portion of the subject proximate the electronic display;
receive, from the optical sensor disposed within the housing opposite the first side of the electronic display, an electronic signal representative of the reflected light off the portion of the subject; and
determine one or more vital signs of the subject based at least in part on the reflected light.

19. The mobile device system of claim 18, wherein no portion of the optical sensor is visible from the first side of the electronic display.

20. The mobile device system of claim 18, wherein the host processor:
receives, from the optical sensor, the electronic signal representative of the reflected light off the portion of the subject in an instance in which the electronic display is illuminated under the portion of the subject, and
determines one or more vital signs of the subject based at least in part on the electronic signal.
